(19) United States
(12) Patent Application Publication
(10) Pub. No.: US 2026/0269022 A1
(43) Pub. Date: Sep. 10, 2026

(54) BIOLOGICAL AGENT AEROSOL CLASSIFICATION/IDENTIFICATION USING MACHINE LEARNING ALGORITHMS

(71) Applicant: Teledyne FLIR Defense, Inc., Thousand Oaks, CA (US)

(72) Inventors: Andrew Eller, West Lafayette, IN (US); Douglas K. Martins, Lafayette, IN (US); William Harman Kenri Casey, West Lafayette, IN (US)

(21) Appl. No.: 19/663,775

(22) Filed: Apr. 30, 2026

Related U.S. Application Data
(63) Continuation of application No. PCT/US2024/054531, filed on Nov. 5, 2024.
(60) Provisional application No. 63/596,946, filed on Nov. 7, 2023.

(51) Int. Cl. G16C 20/20; G06N 20/00; G16B 40/20; G16C 20/70

(52) U.S. Cl. CPC G16C 20/20; G06N 20/00; G16B 40/20; G16C 20/70

(57) ABSTRACT

In accordance with various embodiments, a system and a method for identifying a particle as a bioactive stimulant are provided. The system includes a processor configured to execute machine-readable instructions borne by a non-transitory computer-readable memory device to cause the processor to process one or more steps of the method disclosed herein. The system/method include the steps to: receive a dataset comprising scattered light signals and/or fluorescent light signals of the particle; analyze the dataset using one or more machine learning models, wherein the one or more machine learning models is trained using elastic scattering light intensity data and fluorescent light intensity data of a library of biological molecules; generate a probability score that the particle is bioactive based on the analysis of the dataset; determine, via classification of the probability score, that the particle is bioactive; and/or output a result indicating that the particle is the bioactive stimulant.

### CROSS REFERENCE TO RELATED APPLICATIONS

This application is a continuation of International Patent Application No. PCT/US2024/054531 filed Nov. 5, 2024 and entitled “BIOLOGICAL AGENT AEROSOL CLASSIFICATION/IDENTIFICATION USING MACHINE LEARNING ALGORITHMS,” which claims priority to and the benefit of U.S. Provisional Patent Application No. 63/596,946 filed Nov. 7, 2023 and entitled “BIOLOGICAL AGENT AEROSOL CLASSIFICATION/IDENTIFICATION USING MACHINE LEARNING ALGORITHMS,” all of which are incorporated herein by reference in their entirety.

### FIELD OF INVENTION

Embodiments of the present disclosure relate generally to biological agent aerosol classification/identification and, more particularly, to systems and methods of using artificial intelligence and machine learning algorithms in fluorescence spectroscopy for real time classification/identification in bioaerosol detection.

### BACKGROUND

A wide variety of hazardous substances require sensing and monitoring to protect people and property from harm. In order to identify the presence of these hazardous substances, they first need to be detected. However, there are specific issues with the sensing and identification of substances, such as biological warfare agents (BWAs), and these issues particularly include their detection in the presence of ubiquitous background contamination of both biological origin (e.g., organically-derived dust and plant pollen) and chemical particulates (e.g., fuel exhausts).

Some of the more suitable techniques that can deal with the aforementioned issue include laser-induced fluorescence (LIF) techniques, which can be used to narrow the range of detected particles to those of biological origin. However, it is still possible for the LIF techniques to falsely trigger on benign biological material. Furthermore, a limitation with all currently fielded BWA sensors is the lack of real-time specific classification or identification of the agent detected. Currently, biological samples must be collected and sent to a laboratory for in-depth analysis to identify the agent. Therefore, there is a need for a more advanced technique that can be used to classify, or even uniquely identify, the biological agent detected in real-time, thereby providing immediate information to responders and significantly enhancing the protection of personnel exposed to an attack.

### SUMMARY

In accordance with various embodiments, the disclosure relates to biological agent aerosol classification/identification using machine learning algorithms.

In accordance with one or more embodiments, a computer-based method of identifying a particle as a bioactive stimulant is provided. The method includes executing on a processor one or more steps of: receiving a dataset comprising scattered light signals and/or fluorescent light signals of the particle; analyzing the dataset using one or more machine learning models, wherein the one or more machine learning models is trained using a library of biological molecules annotated via supervised-learning based on elastic scattering light intensity data and fluorescent light intensity data of the biological molecules; generating a probability score that the particle is bioactive based on the analysis of the dataset; and determining, via classification of the probability score, that the particle is bioactive. In one or more embodiments, analyzing the dataset in the method includes identifying, via the one or more machine learning models, one or more features associated with the scattered light signals and/or the fluorescent light signals of the particle; and classifying the particle as a bioactive stimulant based on a ranking of the one or more identified features.

In accordance with various embodiments, a system for identifying a particle as a bioactive stimulant is provided. The system includes a processor configured to execute machine-readable instructions borne by a non-transitory computer-readable memory device to cause the processor to: receive a dataset comprising scattered light signals and/or fluorescent light signals of the particle; analyze the dataset using one or more machine learning models, wherein the one or more machine learning models is trained using elastic scattering light intensity data and fluorescent light intensity data of a library of biological molecules; generate a probability score that the particle is bioactive based on the analysis of the dataset; determine, via classification of the probability score, that the particle is bioactive; and output a result indicating that the particle is the bioactive stimulant.

In accordance with various embodiments, a method of generating a machine learning model for identifying a bioactive particle is provided. The method includes receiving a first dataset comprising elastic scattering light intensity data and fluorescent light intensity data of a first molecule; identifying one or more features associated with the elastic scattering light intensity data and the fluorescent light intensity data; training a machine learning model using the one or more identified features. In one or more embodiments, the training includes performing the machine learning model to produce one or more confidence level associated with the one or more identified features; classifying the identified one or more features based on the one or more confidence level; validating the machine learning model using a second dataset comprising elastic scattering light intensity data and fluorescent light intensity data of a second molecule; and optimizing the machine learning model by modifying the machine learning model using a third dataset comprising elastic scattering light intensity data and fluorescent light intensity data of a third molecule. In various embodiments, the machine learning model includes a random forest classifier model, a K-Nearest Neighbors model, a deep neural network, or a recurrent neural network.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 illustrates a method of identifying a particle as a bioactive stimulant, in accordance with various embodiments.

FIG. 2 illustrates a system for identifying a particle as a bioactive stimulant, in accordance with various embodiments.

FIG. 3 illustrates a method of generating a machine learning model for identifying a bioactive particle, in accordance with various embodiments.

FIG. 4 illustrates a block diagram of a computer system, in accordance with various embodiments.

FIG. 5A illustrates an example machine learning model or a neural network training process, in accordance with one or more embodiments.

FIG. 5B illustrates a validation process for the neural network of FIG. 5A, in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Current disclosure describes one or more embodiments of the systems and methods for real time classification/identification in bioaerosol detection. The embodiments disclosed herein can facilitate with real time classification/identification in bioaerosol detection, thereby providing immediate information to emergency responders and significantly enhancing the protection of personnel exposed to biological warfare agents (BWAs). In particular, one or more embodiments disclosed herein include a system and a method for detecting and identification of bioactive aerosols in a diverse set of environments. In one or more embodiments, the system/method utilizes ultraviolet laser-induced fluorescence (UV-LIF) to continuously monitor a stream of air for fluorescence and characteristic particle size as a bioaerosol trigger. In accordance with one or more embodiments, the disclosed system/method can detect and identify bioaerosol threats in real-time. In accordance with one or more embodiments, the disclosed system/method can be configured for reducing false alarms and improving detection capabilities in noisy urban environments. In one or more embodiments, the disclosed system/method can serve as an early-warning sensor that collects a stream of air onto a membrane for offline confirmatory analysis.

In accordance with one or more embodiments, the disclosed system/method include the use of machine-learning based approaches for providing real-time identification of bioaerosols. In accordance with one or more embodiments, the disclosed system/method can include detection of a plurality of features to train the machine-learning models. In accordance with one or more embodiments, the disclosed system/method can include testing and comparing of the models to determine one or more high performing models. In accordance with one or more embodiments, a high performing model includes a Random Forest Classifier (RFL), a K Nearest Neighbors (KNN), or an Artificial Neural Network (ANN) model, which helps with positive identifications of a bioactive simulant of greater than 90% and a near-zero false alarm rate.

In accordance with one or more embodiments, the use of machine-learning algorithms in the disclosed system/method can improve the capability of the data collection, thereby improving the classification and identification of the biological agents being detected, rather than simply providing a trigger, or indication, that some form of biological agent is present. In various embodiments, the disclosed system/method utilizes an optical cell that is configured to generate data for the machine-learning models described in this disclosure. The construction of the optical cell is described in U.S. Pat. No. 6,885,440 B2: System and method for detecting and classifying biological particles, and U.S. Pat. No. 7,106,442 B2: Multi-spectral optical method for detecting and classifying biological and non-biological particles, the contents of which are hereby incorporated by reference in their entirety.

The detection algorithm described in this disclosure is a machine-learning based classification and identification algorithm for bioaersol detection systems and methodologies. Once an optical cell, e.g., a cell in the UV-LIF range, generates data through two channels: a photomultiplier detector that measures the intensity of the incident laser (elastic scatter) light and a photomultiplier detector that measures the intensity of wavelengths >405 nm (fluorescent signal). The data sorting algorithm on the system registers and bins this information relative to size and fluorescent thresholds set in calibration. In total, the system generates 10+ data features from these channels that can be incorporated into a decision-making algorithm. The fluorescent data generated is used to determine if a particular target particle is bioactive while the elastic scattering data is used to sort particles by size and provide an estimated particle count, in accordance with one or more disclosed embodiments.

Among the machine-learning models used in the system and method herein are RFL, KNN and ANN models. In some embodiments, the RFL algorithm works by treating each datapoint a set of parallel decision trees and averaging the results to calculate a final prediction. The KNN operates by plotting each datapoint and measuring a distance value to each different class in the model; it returns a prediction based on the class with the most data points at the lowest distance. The ANN processes data through a densely connected neural network that calculates non-linear patterns to resolve a prediction. All three approaches have been reliable and high confident and any combination of these three may be utilized in the disclosed system and method described herein.

In various embodiments, the machine-learning algorithm incorporates these features to build a trained library of biological molecules through supervised learning in a process described as follows. To train the system on a new agent or simulant, the operator exposes the system to the stimulus then saves the resultant data file. The file is transferred to a computer that houses the algorithm for training. Each result file contains fluorescent and elastic scattering light intensity information for each second of operation as these points are specific to the stimulus tested, they are incorporated into the training set. Thus a 60-second data file generates 60 useable training datapoints.

In one or more embodiments, the machine-learning model can be applied to a datastream as it is being collected by the system. For example, in one embodiment, each second that a data point is generated, it can be passed to the model in near real-time to output a prediction. In one or more embodiments, the data analysis by the machine-learning model on the data from the datastream is being done using a discrete machine-learning capable process, for example, in a computer. In one or more embodiments, the data analysis being applied to the machine-learning is capable of classifying/identifying one or more bioactive simulants with a high confidence and with low false alarm rates.

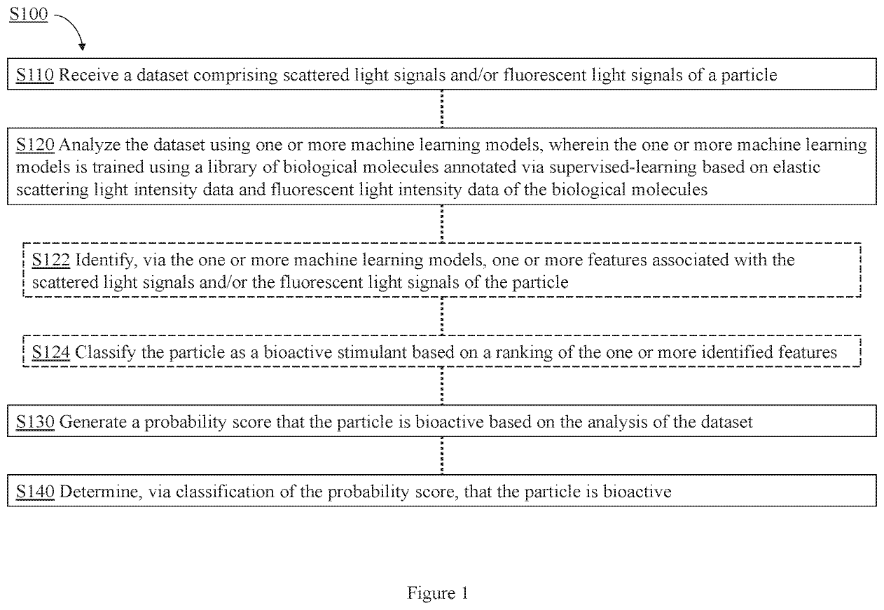

The disclosed system and methods are further described with respect to FIGS. 1-5, in accordance with various embodiments.

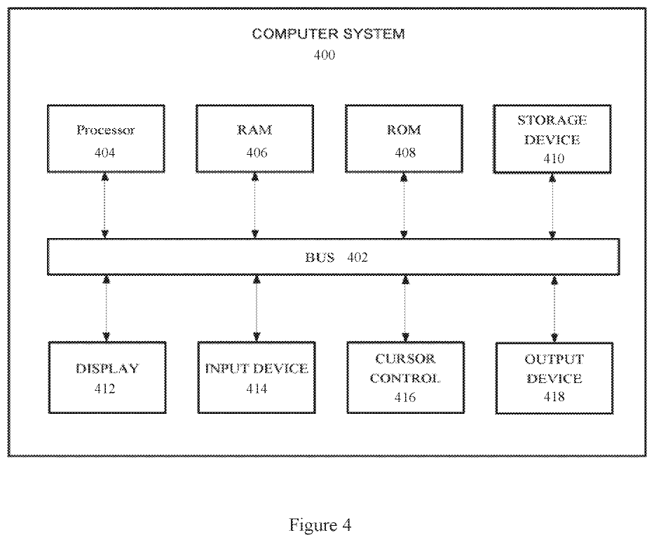

FIG. 1 illustrates a method S100 of identifying a particle as a bioactive stimulant, in accordance with various embodiments. In one or more embodiments, the method S100 is a computer-based method that can be executed on a processor of a computer, such as the computer system described with respect to FIG. 4. The method S100 includes, at step S110, receiving a dataset comprising scattered light signals and/or fluorescent light signals of the particle. In various embodiments, the dataset can include, for example, but not limited to data acquired or received in a period of one second, two seconds, three seconds, or any number of seconds or minutes, or hours of operation, as these points can be specific to the stimulus tested, they are incorporated into the training set. In some embodiments, the dataset includes data points obtained within a period of one second. In some embodiments, the dataset includes data points obtained over a portion or an entirety of the dataset. In some embodiments, a one-second dataset can be used to generate one useable training datapoint, whereas a 60-second dataset can be used to generate 60 useable training datapoints.

As illustrated in FIG. 1, the method S100 also includes, at step S120, analyzing the dataset using one or more machine learning models. In one or more embodiments, the one or more machine learning models can include a random forest classifier model, a K-Nearest Neighbors model, a deep neural network, or recurrent neural network. In one or more embodiments, the one or more machine learning models can be trained using a library of biological molecules annotated via, for example but not limited to supervised-learning, based on elastic scattering light intensity data and fluorescent light intensity data of the biological molecules. In one or more embodiments, the library of biological molecules includes one or more dry agents in a form of a powder. In one or more embodiments, the library of biological molecules includes one or more wet agents in a form of a powder dissolved in water.

In one or more embodiments, the dry agents can include B12, baby powder, baking powder, baking soda, yeast, flour, milk powder, Niacin, paprika, pineapple gelatin, miso, or turmeric, among many others. In one or more embodiments, the wet agents can include B12, baby powder, baking powder, baking soda, yeast, flour, milk powder, Niacin, paprika, pineapple gelatin, or miso, dissolved in water.

In one or more embodiments of the method S100, analyzing the data of S120 further includes, optionally at Step S122, identifying, via the one or more machine learning models, one or more features associated with the scattered light signals and/or the fluorescent light signals of the particle; and optionally at Step S124, classifying the particle as a bioactive stimulant based on a ranking of the one or more identified features. In various embodiments, the one or more identified features include ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, or ‘bpct_tot_a’. Table 1 below shows the identified features associated with the scattered light signals and/or the fluorescent light signals as follows:

TABLE 1

| Abbreviation | Definition |
|---|---|
| c_s_i | Instantaneous measure of the elastic scatter of small particles |
| c_s_a | Average measure of the elastic scatter of small particles |
| c_l_i | Instantaneous measure of the elastic scatter of large particles |
| sfl_a | Average measure of the size fraction of large particles |
| bc_s_i | Instantaneous measure of the fluorescence of small particles |
| bc_s_a | Average measure of the fluorescence of small particles |
| bc_l_i | Instantaneous measure of the fluorescence of large particles |
| bc_l_a | Average measure of the fluorescence of large particles |
| bpct_s_i | Instantaneous measure of the percent of small particles that are |
| | biological |
| bpct_s_a | Average measure of the percent of small particles that are biological |
| bpct_l_i | Instantaneous measure of the percent of large particles that are biological |
| bpct_l_a | Average measure of the percent of large particles that are biological |
| bpct_tot_i | Instantaneous measure of the percent of all particles that are biological |
| bpct_tot_a | Average measure of the percent of all particles that are biological |

In one or more embodiments, the one or more features shown in Table 1 may be interrelated. For example, “bc_s” is a number of bio-fluorescent events that correlate with a small particle, while “bc_l” is the number of bio-fluorescent events that correlate with large particles, and the “bpct” is the percentage of particles that were size small or large, respectively.

In one or more embodiments, at least 3 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, “bpct_l_a”, ‘bpct_tot_i’, and ‘bpct_tot_a’ are used in the analysis. In one or more embodiments, at least 5 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, at least 10 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis step of S120.

In one or more embodiments, no more than 3 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, no more than 5 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, no more than 10 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, “bpct_l_i”, ‘bpct_l_a’, ‘bpct_tot_i’, and ‘bpct_tot_a’ are used in the analysis step of S120.

As illustrated in FIG. 1, the method S100 also includes, at step S130, generating a probability score that the particle is bioactive based on the analysis of the dataset, and at step S140, determining, via classification of the probability score, that the particle is bioactive, in accordance with various embodiments.

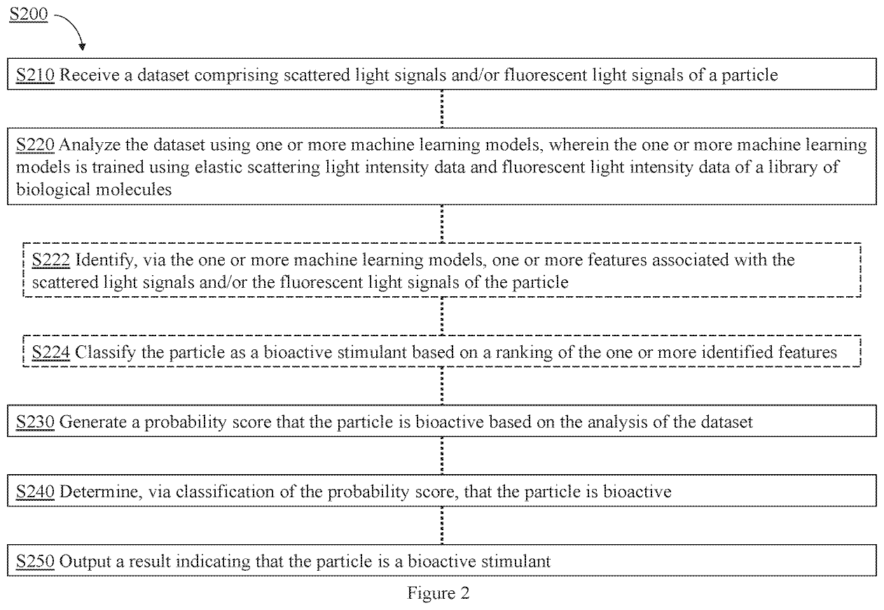

Now referring to FIG. 2, which illustrates a system S200 for identifying a particle as a bioactive stimulant, in accordance with various embodiments. In one or more embodiments, the system S200 includes a processor configured to execute machine-readable instructions borne by a non-transitory computer-readable memory device. The processor can be a computer, such as the computer system described with respect to FIG. 4. In accordance with one or more embodiments, the machine-readable instructions cause the processor of the system S200 to receive a dataset comprising scattered light signals and/or fluorescent light signals of the particle, at step S210; analyze the dataset using one or more machine learning models, wherein the one or more machine learning models is trained using elastic scattering light intensity data and fluorescent light intensity data of a library of biological molecules, at step S220; generate a probability score that the particle is bioactive based on the analysis of the dataset, at step S230; determine, via classification of the probability score, that the particle is bioactive, at step S240; and output a result indicating that the particle is the bioactive stimulant, at step S250.

As further illustrated in FIG. 2, analyzing the dataset at step S220 further includes executing additional machine-readable instructions cause the processor of the system S200 to, optionally at step S222, identify, via the one or more machine learning models, one or more features associated with the scattered light signals and/or the fluorescent light signals of the particle; and optionally at step S224, classify the particle as a bioactive stimulant based on a ranking of the one or more identified features.

In various embodiments, the one or more identified features used in the one or more machine learning models implemented in the system S200 can include ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, or ‘bpct_tot_a’. In one or more embodiments, at least 3 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, at least 5 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, at least 10 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, “bpct_s_i”, ‘bpct_s_a’, ‘bpct_l_i’, ‘bpct_l_a’, ‘bpct_tot_i’, and ‘bpct_tot_a’ are used in the analysis step of S220.

In one or more embodiments, no more than 3 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis step of S220. In one or more embodiments, no more than 5 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis step of S220. In one or more embodiments, no more than 10 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis step of S220.

In one or more embodiments, the dataset can include data points obtained within a period of one second. In one or more embodiments, the one or more trained machine learning models used in the analysis can include a random forest classifier model, a K-Nearest Neighbors model, or a deep neural network. In one or more embodiments, the dataset can include data points obtained over a portion or an entirety of the dataset. In one or more embodiments, the one or more trained machine learning models used in the analysis can include a recurrent neural network.

In one or more embodiments, the library of biological molecules used in the system S200 can include one or more dry agents in a form of a powder. In one or more embodiments, the library of biological molecules used in the system S200 can include includes one or more wet agents in a form of a powder dissolved in water. In one or more embodiments, the dry agents can include B12, baby powder, baking powder, baking soda, yeast, flour, milk powder, Niacin, paprika, pineapple gelatin, miso, or turmeric, among many others. In one or more embodiments, the wet agents can include B12, baby powder, baking powder, baking soda, yeast, flour, milk powder, Niacin, paprika, pineapple gelatin, or miso, dissolved in water.

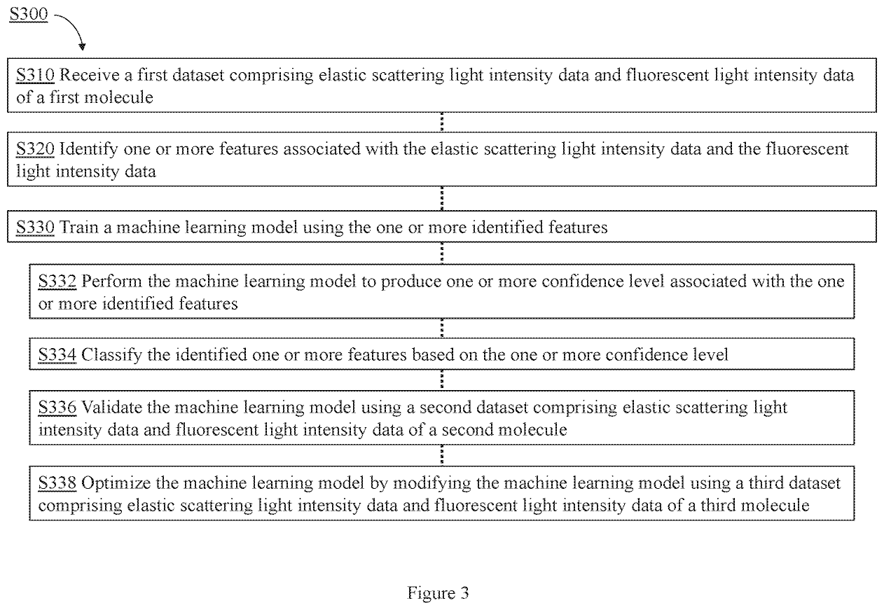

FIG. 3 illustrates a method S300 of generating a machine learning model for identifying a bioactive particle, in accordance with various embodiments. In one or more embodiments, the method S300 is a computer-based method that can be executed on a processor of a computer, such as the computer system described with respect to FIG. 4. The method S300 includes, at step S310, receiving a first dataset comprising elastic scattering light intensity data and fluorescent light intensity data of a first molecule; at step S320, identifying one or more features associated with the elastic scattering light intensity data and the fluorescent light intensity data; and at step S330, training a machine learning model using the one or more identified features, in accordance with one or more embodiments herein. In some embodiments, the training step of S330 further includes optionally, at step S332, performing the machine learning model to produce one or more confidence level associated with the one or more identified features; at step S334, classifying the identified one or more features based on the one or more confidence level; at step S336, validating the machine learning model using a second dataset comprising elastic scattering light intensity data and fluorescent light intensity data of a second molecule; and at step S338, optimizing the machine learning model by modifying the machine learning model using a third dataset comprising elastic scattering light intensity data and fluorescent light intensity data of a third molecule.

In various embodiments of the method S300, the machine learning model may include a random forest classifier model, a K-Nearest Neighbors model, a deep neural network, or a recurrent neural network. In various embodiments, the one or more identified features include ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, “bpct_s_a”, ‘bpct_l_i’, ‘bpct_l_a’, ‘bpct_tot_i’, or ‘bpct_tot_a’. In one or more embodiments, at least 3 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, at least 5 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis. In one or more embodiments, at least 10 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the classifying step of S334.

In one or more embodiments, no more than 3 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis step of S334. In one or more embodiments, no more than 5 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, “bpct_s_i”, ‘bpct_s_a’, ‘bpct_l_i’, ‘bpct_l_a’, ‘bpct_tot_i’, and “bpct_tot_a” are used in the analysis step of S334. In one or more embodiments, no more than 10 of the features from the list of ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, and ‘bpct_tot_a’ are used in the analysis step of S334.

In various embodiments, the first, second, or third dataset comprises data points obtained within a period of one second, or data points from a portion or an entirety of the respective dataset.

FIG. 4 is a block diagram of a computer system 400, in accordance with various embodiments. Computer system 400 may be used as a processor for the system S200 as described with respect to FIG. 2 and methods S100 and S300, as described with respect to FIGS. 1 and 3, respectively.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

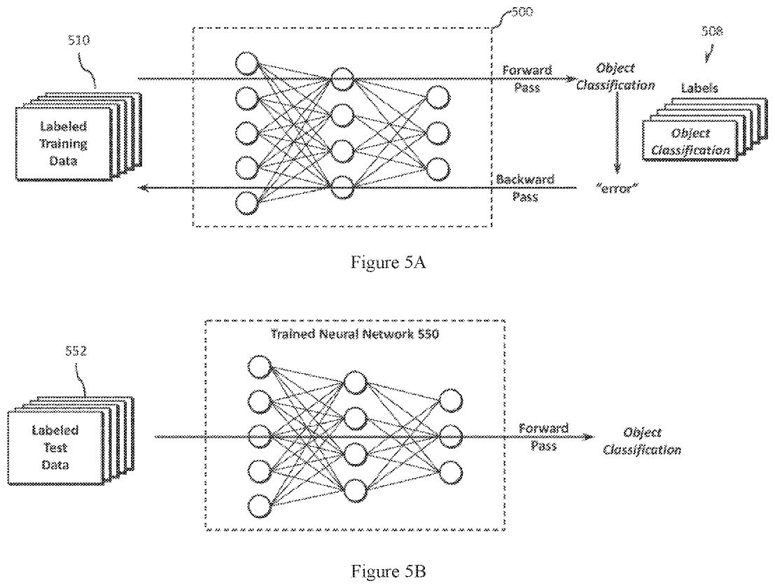

Referring to FIG. 5A, an example a machine learning model or a neural network that may be used to generate trained training models are described, in accordance with one or more embodiments. The neural network 500 is implemented as a recurrent neural network, artificial neural network or other suitable neural network that receives a labeled training dataset 510 to produce object detection information 508 for each data sample. The training dataset includes elastic scattering light intensity data and fluorescent light intensity data of a plurality of biological molecules. For object classification, the dataset may include fluorescent light intensity data that include an object (e.g., a particle) to be identified.

The training includes a forward pass through the neural network 500 to produce object detection and classification information, such as an object type, an object classification, and a confidence level in the object classification. Each data sample is labeled with the correct classification and the output of the neural network 500 is compared to the correct label. If the neural network 500 mislabels the input data, then a backward pass through the neural network 500 may be used to adjust the neural network to correct for the misclassification. Referring to FIG. 5B, a trained neural network 550, may then be tested for accuracy using a set of labeled test data 552. The trained neural network 550 may then be implemented in a processor to detect and classify objects.

In one or more embodiments, the trained neural network 550 may be used, for example, with the method S100, the system S200, and the method S300 as described respectively with FIGS. 1, 2, and 3. In some embodiments, the trained neural network 550 can be implemented in the analysis step of the method S100, the system S200, and the method S300 to generate a probability score that the particle is bioactive; determine based on classification of the probability score, that the particle is bioactive; and respectively, output a result indicating that the particle is the bioactive stimulant. Based on this disclosure, the various embodiments of methodologies and system described with respect to FIGS. 1-5 can help greatly in classifying, or uniquely identifying, the biological agent or particle in any environment and include in real-time applications.

What is claimed is:

1. A computer-based method of identifying a particle as a bioactive stimulant, the method comprising executing on a processor the steps of:
receiving a dataset comprising scattered light signals and/or fluorescent light signals of the particle;
analyzing the dataset using one or more machine learning models, wherein the one or more machine learning models is trained using a library of biological molecules annotated via supervised-learning based on elastic scattering light intensity data and fluorescent light intensity data of the biological molecules;
generating a probability score that the particle is bioactive based on the analysis of the dataset; and
determining, via classification of the probability score, that the particle is bioactive.

2. The method of claim 1, wherein analyzing the dataset comprises:
identifying, via the one or more machine learning models, one or more features associated with the scattered light signals and/or the fluorescent light signals of the particle; and
classifying the particle as a bioactive stimulant based on a ranking of the one or more identified features.

3. The method of claim 1 or 2, wherein the dataset comprises data points obtained within a period of one second.

4. The method of claim 3, wherein the one or more trained machine learning models comprises a random forest classifier model, a K-Nearest Neighbors model, or a deep neural network.

5. The method of any one of claims 1-4, wherein the dataset comprises data points obtained over a portion or an entirety of the dataset.

6. The method of claim 5, wherein the one or more trained machine learning models comprises a recurrent neural network.

7. The method of any one of claims 1-6, wherein the library of biological molecules comprises one or more dry agents in a form of a powder.

8. The method of any one of claims 2-7, wherein the one or more identified features include ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, or ‘bpct_tot_a’.

9. A system for identifying a particle as a bioactive stimulant, the system comprising:
a processor configured to execute machine-readable instructions borne by a non-transitory computer-readable memory device to cause the processor to:
receive a dataset comprising scattered light signals and/or fluorescent light signals of the particle;
analyze the dataset using one or more machine learning models, wherein the one or more machine learning models is trained using elastic scattering light intensity data and fluorescent light intensity data of a library of biological molecules;
generate a probability score that the particle is bioactive based on the analysis of the dataset;
determine, via classification of the probability score, that the particle is bioactive; and
output a result indicating that the particle is the bioactive stimulant.

10. The system of claim 9, wherein analyzing the dataset comprises instructions to cause the process to:
identify, via the one or more machine learning models, one or more features associated with the scattered light signals and/or the fluorescent light signals of the particle; and
classify the particle as a bioactive stimulant based on a ranking of the one or more identified features.

11. The system of claim 9 or 10, wherein the dataset comprises data points obtained within a period of one second.

12. The system of claim 11, wherein the one or more trained machine learning models comprises a random forest classifier model, a K-Nearest Neighbors model, or a deep neural network.

13. The system of any one of claims 9-12, wherein the dataset comprises data points obtained over a portion or an entirety of the dataset.

14. The system of claim 13, wherein the one or more trained machine learning models comprises a recurrent neural network.

15. The system of any one of claims 9-14, wherein the library of biological molecules comprises one or more dry agents in a form of a powder.

16. The method of any one of claims 10-15, wherein the one or more identified features include ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, or ‘bpct_tot_a’.

17. A method of generating a machine learning model for identifying a bioactive particle, comprising:
receiving a first dataset comprising elastic scattering light intensity data and fluorescent light intensity data of a first molecule;
identifying one or more features associated with the elastic scattering light intensity data and the fluorescent light intensity data;
training a machine learning model using the one or more identified features, wherein the training comprises:
performing the machine learning model to produce one or more confidence level associated with the one or more identified features;
classifying the identified one or more features based on the one or more confidence level;
validating the machine learning model using a second dataset comprising elastic scattering light intensity data and fluorescent light intensity data of a second molecule; and
optimizing the machine learning model by modifying the machine learning model using a third dataset comprising elastic scattering light intensity data and fluorescent light intensity data of a third molecule.

18. The method of claim 17, wherein the machine learning model comprises a random forest classifier model, a K-Nearest Neighbors model, a deep neural network, or a recurrent neural network.

19. The method of claim 17 or 18, wherein the one or more identified features include ‘c_s_i’, ‘c_s_a’, ‘c_l_i’, ‘c_l_a’, ‘sfl_a’, ‘bc_s_i’, ‘bc_s_a’, ‘bc_l_i’, ‘bc_l_a’, ‘bpct_s_i’, ‘bpct_s_a’, ‘bpct_l_i’, ‘bpct_l_a’, ‘bpct_tot_i’, or ‘bpct_tot_a’.

20. The method of any one of claims 17-19, wherein the first, second, or third dataset comprises data points obtained within a period of one second, or data points from a portion or an entirety of the respective dataset.
